(19) United States
(12) Patent Application Publication
(10) Pub. No.: US 2026/0057980 A1
(43) Pub. Date: Feb. 26, 2026

(54) REMOVABLE PATIENT HEALTH INFORMATION ON MEDICAL DEVICE PACKAGING

(71) Applicant: Starkey Laboratories, Inc., Eden Prairie, MN (US)

(72) Inventors: Brian Thomas Dahl, Minnetrista, MN (US); Scott Sanderson, Minneapolis, MN (US); Jessica Ehrlich, Minneapolis, MN (US)

(21) Appl. No.: 18/810,135

(22) Filed: Aug. 20, 2024

(51) Int. Cl. G16H 15/00; A61B 90/94

(52) U.S. Cl. CPC G16H 15/00; A61B 90/94

(57) ABSTRACT

The present subject matter relates to a packaging system and related methods for medical devices or accessories that enhance the management and security of patient health information. The system includes a package containing a removable element designed to be easily detached without the use of tools, featuring patient health information indicia. These indicia may include sensitive data such as a patient's name and social security number. The removable element can be designed with rounded corners and a series of perforations for easy removal. Additionally, the package may include a void next to an edge of the removable element to help initiate its removal. Once removed, the removable element can be attached to a carrying case for the medical device or accessory using a fastening means or placed into a built-in sleeve, serving as a continuous reference for patient and device or accessory information.

### TECHNICAL FIELD

This patent document pertains to medical devices and accessories. More particularly, but not by way of limitation, the patent document pertains to a packaging system for medical devices and accessories that incorporates patient health information.

### BACKGROUND

In recent years, the healthcare industry has seen an increased focus on patient privacy and information security, particularly with the implementation of regulations such as the Health Insurance Portability and Accountability Act (HIPAA) in the United States. These regulations have significantly impacted various aspects of healthcare delivery.

One of the largest healthcare providers in the United States has implemented specific requirements for hearing aid manufacturers to attach a ‘patient label’ to the package of each hearing aid order. This label, containing the patient's name and the last four digits of their Social Security Number (SSN), serves as an inventory tracking tool within the facilities. The primary purpose of this labeling system is to optimize workflows, ensuring that any audiologist can quickly locate the correct order and efficiently fit the patient with their prescribed hearing device.

However, while beneficial for inventory management and patient care, this system has inadvertently created a new challenge. The patient information on these labels is considered Protected Health Information (PHI) under HIPAA regulations. Consequently, audiologists are required to remove and securely dispose of these labels by the end of each day to maintain patient privacy and comply with HIPAA guidelines.

This additional task of cutting off and shredding the patient labels from hearing aid packaging has become a significant burden for audiologists. Given the time-sensitive nature of their work and the high volume of patients they serve, this extra step reduces the time available for patient care and device fittings. The problem is compounded by the fact that audiologists are already operating under tight time constraints, and any reduction in their available time for patient care can have a ripple effect on overall service quality and efficiency.

### SUMMARY

Current practices in the hearing aid industry have not adequately addressed the issue of requiring audiologists to cut off and shred patient labels. Manufacturers continue to use permanently affixed labels on their packaging, forcing healthcare providers to undertake the time-consuming removal process. While this approach satisfies inventory tracking requirements, such as those of the Veterans Affairs (VA) healthcare system, it fails to consider the downstream implications for healthcare providers and their workflow efficiency.

The present inventors have recognized the need for a solution that effectively balances the requirements for inventory tracking, patient privacy, and clinical efficiency. They further recognized that a solution would retain the advantages of existing labeling systems while reducing the time and effort needed for label removal and disposal. Moreover, they recognized that such a solution should be versatile enough to serve multiple purposes, thereby enhancing its value in clinical settings.

The present subject matter introduces an innovative packaging system for ear-wearable devices or accessories that enhances both user experience and security by integrating patient health information into the packaging. This system includes a package that can enclose an ear-wearable device or accessory and features a removable element with patient health information indicia. This removable element is designed for easy detachment from the package without the need for tools. User-friendly features, such as a void adjacent to an edge of the removable element or a pull tab, rounded corners, and a series of perforations, facilitate its removal.

Additionally, the removable element can serve a dual purpose. Beyond its role as a product identifier for inventory management when attached to the package, it can be reattached to another surface or secured to a provided carrying case for the ear-wearable device or accessory, serving as a separate information card after its removal. The dimensions of the removable element can be designed for convenient handling, measuring approximately 2.5 to 8 centimeters in width and 5 to 13 centimeters in length.

This patent document also encompasses methods related to the use and management of the packaging system, focusing on the application and utility of the removable element both as a product identifier when attached to the package and as a standalone information card post-removal. These methods provide a structured approach to managing patient health information while maintaining the integrity and functionality of the packaging for ear-wearable and other medical devices.

These and other examples and features of the present systems and methods will be described, at least in part, in the following Detailed Description. This Overview offers non-limiting examples of the present subject matter and is not intended to provide an exclusive or exhaustive explanation. The Detailed Description provided below is included to offer additional insights into the present systems and methods.

### BRIEF DESCRIPTION OF THE FIGURES

In the drawings, similar features and components are represented by like numerals across the various views. The drawings illustrate generally, by way of example, but not by way of limitation, various system and method embodiments discussed in this patent document.

FIG. 1 is a perspective view of a medical device packaging system including a package, a carrying case housed within the package, and a removable element adorned with patient health information indicia, as constructed in accordance with at least one embodiment.

FIGS. 2A-2D are perspective views of a packaging system during a method for detaching a removable element from a package without the use of tools, as constructed in accordance with at least one embodiment.

FIG. 3 is a perspective view of a package with a sleeve having a transparent outward-facing surface designed to accommodate a removable element in the form of a patient health information card, as constructed in advance with at least one embodiment.

FIG. 4 is a perspective view of how a removable element can be secured to a carrying case once removed from a package, as constructed in accordance with at least one embodiment.

FIG. 5 is a top plan view of dimensional details of a package and its removable element, highlighting the removable element's practical size and handling ease, as constructed in accordance with at least one embodiment.

FIG. 6 is a perspective view of a tear-off portion of indicia affixed to a removable element, displaying the element before the removal of sensitive information, as constructed in accordance with at least one embodiment.

FIG. 7 is a flowchart showing the sequential use of a packaging system, from initial packaging through the potential secure disposal or repurposing of the removable element, as constructed in accordance with at least one embodiment.

The drawings are not necessarily to scale. Some features and components may be shown exaggerated in scale or represented in schematic form for clarity and conciseness, and certain details may be omitted.

### DETAILED DESCRIPTION

The present subject matter relates to an innovative packaging system for ear-wearable devices or accessories, designed to transform patient information management and improve efficiency in audiological practices. The packaging portion of this system comprises three core components: a package for an ear-wearable device or accessory, a removable element that is strategically integrated into the package, and patient health information indicia that are affixed to the removable element.

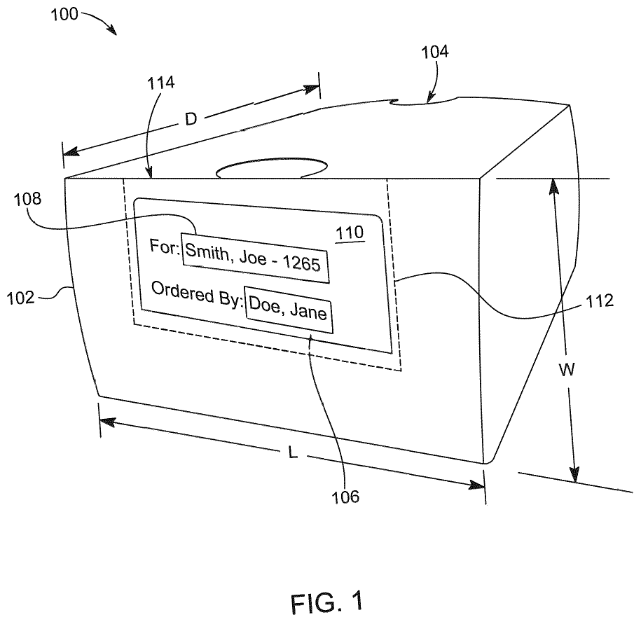

FIG. 1 provides an overview of a medical device packaging system (100), showcasing a package (102). This package (102) securely encases a carrying case (104) that houses an ear-wearable device or accessory, which could be items such as hearing aids, cochlear implants, or associated accessories like remote controls or charging units.

In some examples, the package (102) can be made from materials like carboard, paper, paperboard, fiberboard, or various polymers and plastics such as high-density polyethylene (HDPE), Mylar®, Tyvek®, or polyester. It might also include composites and combinations of these materials, as well as other suitable packaging materials. Additionally, the package (102) may consist of multiple layers incorporating different materials. The overall dimensions—length (L), width (W), and depth (D)—of the package (102) can be varied or adjusted as necessary to fit various products that it may contain.

The packaging system (100) includes a removable element (106) integrated with the package (102). This element (106) carries patient health information indicia (108), which could be printed directly on it or on a label (110) affixed to it. The patient health information indicia (108) can include the patient's name and the last four digits of their SSN to facilitate inventory tracking. The indicia (108) may also contain additional, non-sensitive details, such as the ordering audiologist's name, hospital or clinic information, instructions for the device or accessory, regulatory compliance data, battery replacement guidelines, and manufacturer's details.

FIG. 1 also highlights the user-friendly design of the removable element (106), including perforations (112) that simplify its removal. Optionally, a backside surface (114) of the removable element (106) can include adhesive or hook and loop fasteners for attachment to another surface after its removal from the package (102). When attached to the package (102), the removable element (106) acts as a readable product identifier that supports efficient inventory management and quick patient association in clinical settings. Once detached, the removable element (106) can transform into a separate information card that the patient can retain, thus enhancing the continuity of care. This dual functionality illustrates the packaging system's (100) innovative approach to merging accessibility of information with security and patient privacy. Alternatively, for situations where the patient's health information is no longer needed, the detached removable element (106) can be shredded or otherwise destroyed.

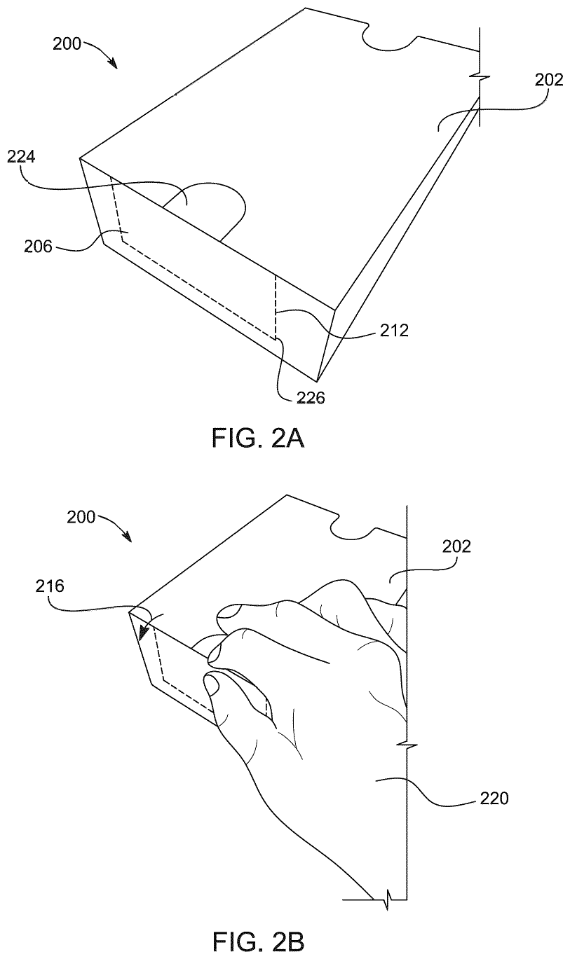

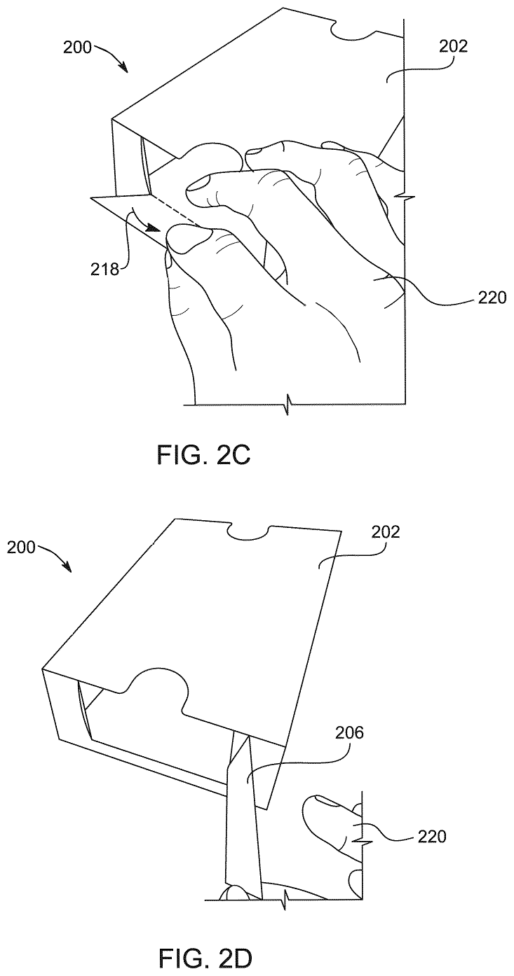

FIGS. 2A-2D showcase illustrations of a removable element (206) within a packaging system (200), specifically designed for tool-less detachment. Arrows (216, 218) in FIGS. 2C and 2D show the direction in which an audiologist's hands (220) might pull to detach the element (206), highlighting how easily it can be removed.

The design of the removable element (206) prioritizes ease of use and functionality. It features a series of precision-engineered perforations (212) that are strategically positioned to allow for a clean and easy tear while preserving the overall structural integrity of the package (202) until removal is initiated. These perforations (212) range from 2 to 4 millimeters, inclusive, in length, with a depth of 50 to 70%, inclusive, of the element's thickness, in some embodiments. The depth can be uniform across all perforations (212) to ensure tear consistency. The spacing between each perforation (212) is approximately 20 to 40%, inclusive, of the perforation length, contributing to a balance between ease of tearing and maintaining structural integrity. The perforations (212) can be arranged in a rectangular/linear pattern, which facilitates a clean removal. These perforations (212) can be made using laser cutting, die cutting, or other suitable techniques to ensure accuracy and consistency across all production batches.

To aid the detachment process, the package (202) design can include a strategically placed void (224) near an edge of the removable element (206). This void (224) can act as an initial grip point, simplifying the starting step of the removal process, which is particularly beneficial for individuals in a hurry. Alternatively, the removable element (206) can be equipped with an ergonomically designed pull tab, which may be made from a slightly more rigid material or feature a textured surface to enhance the user's grip. Additionally, the corners (226) of the removable element (206) can be rounded, with a radius of about ⅛-inch (3.175 millimeters) minimum in some embodiments. This design consideration can serve multiple purposes: it can prevent the corners (226) from snagging on the package (202) during removal, minimize the risk of paper cuts, and improve the visual and tactile appeal of the element (206) when it is used independently as an information card. These features can collectively enhance the user experience by making the removable element (206) not only functional but also user-friendly to remove.

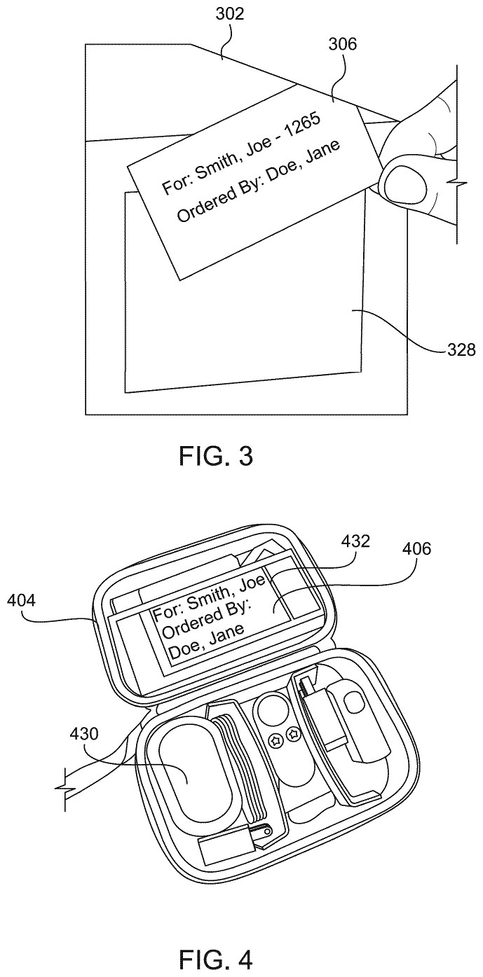

In some alternative examples, as illustrated in FIG. 3, the package (302) incorporates a specialized sleeve (328) with a transparent outward-facing surface, designed to accommodate a removable element (306) configured as a patient health information card. This design feature facilitates the easy viewing of information, allowing the removable element (306) to be viewed and then removed from the sleeve (328) when desired. Made from a durable, clear polymer, the sleeve (328) can ensure that the information on the removable element (306) is legible and preserved over time. In some embodiments, the sleeve (328) includes an open top edge.

FIG. 4 provides a detailed view of how a removable element (406) can attach to a carrying case (404), shown in an open position, provided with the ear-wearable device or accessory (430). This design allows the removable element and the information thereon to be used by the patient at home once it is removed from its package. The illustration shows the element (406) inserted into a sleeve (432) on the carrying case (404), highlighting the element's versatility and practical reusability. The removable element (406) can also be fastened to the carrying case (404) using either an adhesive or fastener backing. The adhesive backing may be specially formulated to retain its stickiness over multiple uses, ensuring its effectiveness doesn't diminish with reattachments. Alternatively, hook and loop fasteners offer a robust, washable, and easy-to-use option that allows for quick detachment and reattachment as needed.

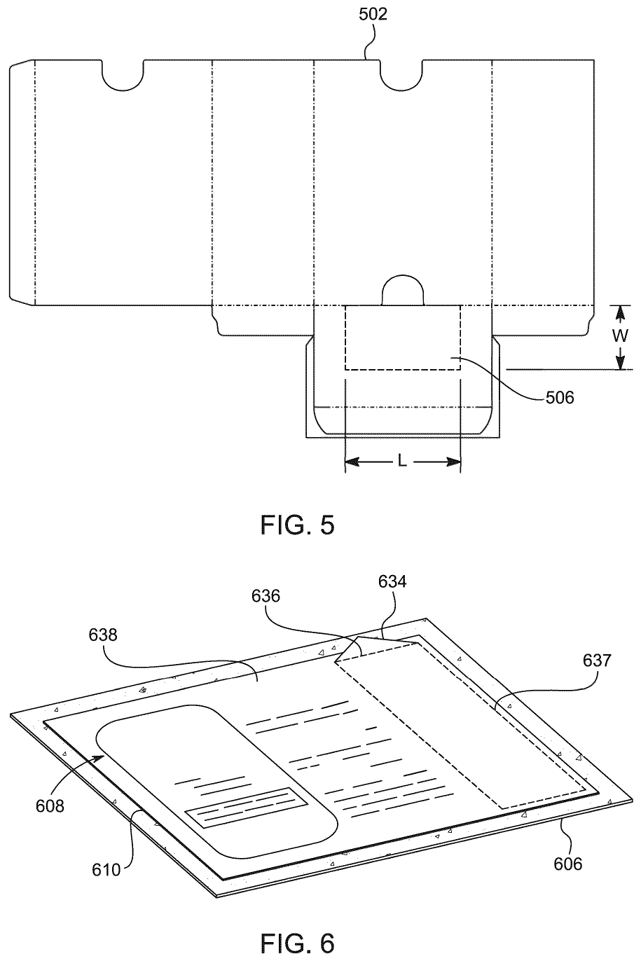

FIG. 5 displays a technical drawing that details the relative dimensions of the package (502) and the removable element (506), including the element's length (L) and width (W). The overall packaging system and the element (506) provide helpful information while remaining compact for easy handling. In varying examples, the width (W) of the removable element (506) ranges from about 2.5 to 8 centimeters, striking a balance between displaying helpful information and facilitating easy handling. Its length (L) varies from about 5 to 13 centimeters. These dimensions balance readability, information capacity, and practical usability.

In some examples, the width (W) of the removable element (506) can be greater than or equal to 2.5 centimeters, 3.0 centimeters, 3.5 centimeters, 4.0 centimeters, 4.5 centimeters, or 5.0 centimeters. In some examples, the width (W) can be less than or equal to 8.0 centimeters, 7.4 centimeters, 6.8 centimeters, 6.2 centimeters, 5.6 centimeters, or 5.0 centimeters. In some examples, the width (W) can fall within a range of 2.5 centimeters to 8.0 centimeters, or 3.0 centimeters to 7.4 centimeters, or 3.5 centimeters to 6.8 centimeters, or 4.0 centimeters to 6.2 centimeters, or 4.5 centimeters to 5.6 centimeters, or can be about 5.0 centimeters. In some examples, the length (L) of the removable element (506) can be greater than or equal to 5.0 centimeters, 5.6 centimeters, 6.2 centimeters, 6.8 centimeters, 7.4 centimeters, or 8.0 centimeters. In some examples, the length (L) can be less than or equal to 13.0 centimeters, 12.0 centimeters, 11.0 centimeters, 10.0 centimeters, 9.0 centimeters, or 8.0 centimeters. In some examples, the length (L) can fall within a range of 5.0 to 13.0 centimeters, or 5.6 to 12.0 centimeters, or 6.2 to 11.0 centimeters, or 6.8 to 10.0 centimeters, or 7.4 to 9.0 centimeters, or can be about 8.0 centimeters.

The removable element (506) includes patient health information indicia, which are crucial for its function within the medical device packaging system. As shown in this and other figures, these indicia can act as a comprehensive identifier for the medical device or accessory. They can feature vital details designed to meet both patient and clinical needs: the patient's full name in a clear, legible font; the last four digits of the patient's social security number or another unique identifier for verification; customer service contact details, which may include phone numbers and QR codes for online resources; the serial number of the device, helpful for tracking warranties and services; and basic care and maintenance instructions for the device or accessory.

FIG. 6 illustrates a removable element (606) of the packaging system, featuring a tear-off portion (634) that includes patient health information indicia (608). The tear-off portion (634) may be created by perforating (636) part of the removable element (606) or a label (610) attached to it. This design enables sensitive PHI information (637) to be displayed and securely removed when no longer needed, while preserving non-sensitive information (638) for later use by the patient.

The illustration shows the removable element (606) before the tear-off portion (634) is removed, highlighting how sensitive information, such as the patient's name and social security number, can be available for inventory tracking requirements and then securely removed. In some examples, the tear-off portion (634) includes a border with perforations to facilitate easy separation without tools from a remainder or base portion of the removable element (606). Alternatively, the tear-off portion could be replaced with a scratch-off portion.

Both the tear-off and scratch-off portions (634) are designed for durability, capable of withstanding normal handling but easily removed when necessary. This ensures that sensitive information remains protected through its removal at the appropriate time.

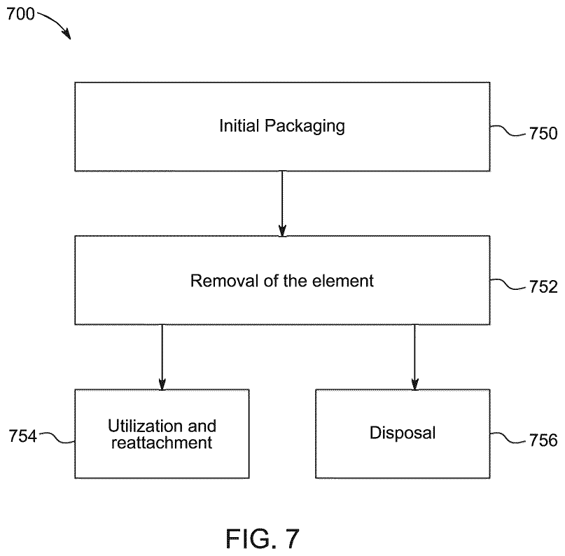

FIG. 7 presents a flowchart 700 that outlines the lifecycle of the packaging system, detailing each stage from initial packaging to the reuse or disposal of the removable element.
1. Initial Packaging: At step 750, the ear-wearable device or accessory is securely encased within the packaging, with the removable element and its indicia prominently displayed and securely attached. This configuration not only protects the device or accessory but also ensures that the accompanying information is immediately accessible for inventory tracking purposes.
2. Removal of the Element: At step 752, the removable element is removed without the need for tools, facilitated by perforations, a pull tab, and/or a strategically placed void. This user-friendly and intuitive design allows for quick access to and removal of the element.
3. Utilization and Reattachment: Following removal, at step 754, the removable element can be attached to a carrying case or used for other purposes relevant to the patient's care routine, thereby enhancing its practical utility beyond its initial packaging purpose.
4. Disposal: At step 756, if the information on the removable element is no longer needed or must be kept confidential, this optional stage outlines methods for secure disposal, such as shredding or other destruction techniques. This ensures that patient information is safely managed and disposed of properly.

The flowchart of FIG. 7 can be modified to accommodate removable elements featuring a tear-off or scratch-off portion, as described in FIG. 6. In this design, some parts of the indicia, including sensitive information, are removed while preserving the non-sensitive parts of the indicia. This allows for the secure handling of sensitive information while maintaining the utility of the non-sensitive information for continued use.

The advantages of adopting the present systems and methods can be considerable. They enhance the accessibility and portability of patient information, securely manage vital data, and improve efficiency in clinical settings—potentially saving audiologists an average of 5 to 7 minutes per patient interaction. Over a typical workday, this could translate into an additional 1 to 2 hours of productive time, allowing for more patient consultations or enhanced quality of care. While the present systems and methods are particularly well-suited for ear-wearable devices like hearing aids, their principles are versatile enough to be adapted for a broad range of medical devices, including glucose monitors, CPAP machines, or other home-use medical devices that require ongoing patient interaction and efficient information management.

The present system also lends itself to specific methods for managing patient health information effectively and efficiently. This involves integrating the removable element during the manufacturing of the package, applying health information indicia prior to dispensing, and configuring the element to serve dual functions—both as a product identifier when attached to the package and as a separate information card when removed. Users can leverage the patient health information indicia as a product identifier, easily remove the element by tearing along the perforations without tools and choose either to securely dispose of it or reattach it to the provided carrying case.

### Closing Notes

The innovative design and functionality of the ear-wearable device or accessory packaging system, as provided in the Detailed Description and illustrated through associated figures, demonstrates significant advancements in medical device management. By integrating a removable element with patient health information indicia into the package, this system enhances the accessibility, portability, and security of crucial patient data. It reduces the time healthcare providers spend per patient interaction and offers practical solutions that improve both patient and provider experiences. Through streamlined processes and a focus on privacy and security, the packaging system plays a vital role in advancing patient care and medical device management.
1The Detailed Description refers to the accompanying drawings, which are an integral part of this description and should be considered in conjunction with it. The drawings illustrate specific embodiments by which the described systems and methods can be implemented, referred to through this document as “examples.”

This Detailed Description is intended to serve as an illustrative guide, not a restrictive one. For instance, the examples described can be combined with each other in various configuration. Additionally, other examples might be apparent to those skilled in the art upon reviewing this document. Moreover, features or components have been grouped in certain instances to simplify the disclosure; however, this does not imply that an unclaimed feature is essential to any claim. It is possible for inventive subject matter to reside in fewer than all features of any given disclosed example. Consequently, the following claim examples are incorporated into this Detailed Description, with each claim example standing as a separate embodiment.

In Example 1, a system for an ear-wearable device or accessory comprises a package, a removable element on the package, and patient health information indicia affixed to the removable element.

In Example 2, the system of Example 1 is configured such that the removable element is detachable from the package without using a tool.

In Example 3, the system of Example 1 or 2 includes a package with a void adjacent to an edge of the removable element, facilitating the initiation of its removal.

In Example 4, the system of Example 1 or 2 features a removable element that includes a pull tab to facilitate its removal from the package.

In Example 5, the system of Examples 1-4 further comprises a perforation border comprising a series of perforations. The perforation border facilitates the removal of the removable element from the package.

In Example 6, the system of Examples 1-5 is designed with rounded corners on the removable element to ease its removal.

In Example 7, the system of Examples 1-6 includes a carrying case for the ear-wearable device or accessory. The removable element is configured to be secured to the carrying case after its removal from the package.

In Example 8, the system of Examples 1-7 further comprises the ear-wearable device or accessory.

In Example 9, the system of Examples 1-8 is configured such that the removable element has an adhesive backing for reattachment to another surface after its removal.

In Example 10, the system of Examples 1-8 features a removable element with a hook or loop fastener backing for reattachment to another surface after its removal.

In Example 11, the system of Examples 1-10 is configured such that the removable element has dimensions of approximately 2.5 to 8 centimeters, inclusive, in width and 5 to 13 centimeters, inclusive, in length.

In Example 12, the system of Examples 1-11 is designed such that the patient health information indicia serve as an ear-wearable device or accessory identifier when affixed to the removable element.

In Example 13, the system of Examples 1-12 is configured such that the removable element comprises a base portion and a separable portion. The base portion comprises a first subset of non-patient health information indicia and the separable portion comprises a second subset of patient health information indicia. The separable portion comprises one of a separable portion perforation border, a peel-off portion, or a scratch-off portion to facilitate removing the second subset of patient health information indicia from the removable element.

In Example 14, the system of Examples 1-13 features a removable element in the form of a patient health information card.

In Example 15, the system of Example 14 is configured such that the patient health information card includes a patient's name or the last four digits of his/her social security number.

In Example 16, the system of Examples 14 or 15 includes customer service contact information for the ear-wearable device on the patient health information card.

In Example 17, the system of Examples 14-16 includes the serial number of the ear-wearable device or accessory on the patient health information card.

In Example 18, the system of Examples 14-17 includes a package with a sleeve featuring a transparent outward-facing surface, designed to hold the patient health information card.

In Example 19, an ear-wearable system comprises a package for the ear-wearable device, a removable element integrated into the package, patient health information indicia affixed to the removable element, and a perforated border around a portion of the removable element to facilitate its removal.

In Example 20, the system of Example 19 includes a carrying case for the ear-wearable device. The removable element is configured to be secured to the carrying case after its removal from the package.

In Example 21, the system of Examples 19 or 20 also includes the ear-wearable device in the form of a hearing aid.

In Example 22, the system of Examples 19-21 features patient health information indicia that include at least one of the following: the patient's name, a customer service contact number for the ear-wearable device, or the ear-wearable device serial number.

In Example 23, a method for managing patient health information for a medical device involves incorporating a removable element into a package for the medical device, applying patient health information indicia to the removable element, and configuring the removable element to serve as a product identifier when attached to the package and as a separate information card when removed.

In Example 24, the method of Example 23 further includes perforating a perforation border of the removable element with a series of perforations to facilitate its removal from the package.

In Example 25, a method for using a packaging system for an ear-wearable device involves using patient health information indicia affixed to a removable element integrated into the package as a product identifier, removing the removable element from the package by tearing along a series of perforations surrounding the removable element, and either shredding the removable element or affixing it to a provided carrying case.

In Example 26, the system or method of any one or any combination of Examples 1-25 can optionally be configured such that all components or options recited are available for use or selection.

Throughout this patent, specific terms are used to refer to particular features or components. It is understood by those skilled in the art that different terms may be used to describe the same feature or component by different individuals or references. This document does not intend to differentiate between components or features that have different names but serve the same function.

In this patent document, specific definitions apply to certain terms unless otherwise specified within the document. The articles “a,” “an,” and “the” include both singular and plural references unless indicated otherwise. For instance, “a component” can mean one component or multiple components. The term “or” is used in a nonexclusive sense, meaning that “A or B” can include situations where either “A”or “B,”or both “A and B,”are present.

All numeric values mentioned are presumed to be prefaced by the word “about” or “approximately,” implying a range that one skilled in the art would consider equivalent to the stated value in terms of function or result. The terms “about” and “approximately” typically encompass variations that are rounded to the nearest significant figure. When numerical ranges are specified by their endpoints, this includes all numbers falling within that range as well as any sub-ranges derived from those endpoints, such as “1 to 4” covering 1, 1.5, 1.75, and so forth, including ranges like “1 to 2” and “2 to 3.5.”

The scope of the present systems and methods should be determined by the appended claims and the full range of equivalents entitled to such claims. In these claims, the terms “including” and “in which” are used as plain-English equivalents to “comprising” and “wherein,” respectively. Furthermore, the terms “including” and “comprising” are open-ended; this means that a device or method that contains additional features or components beyond those listed after such terms in a claim still falls within the scope of that claim. Additionally, in these claims, the labels “first,” “second,” “third,” etc., are used solely as identifiers and do not impose numerical requirements on their objects.

The Abstract is provided to enable readers to quickly understand the nature of the technical disclosure. It is submitted with the understanding that it will not be used to interpret or limit the scope or meaning of the claims.

1. An ear-wearable device or accessory system, comprising:
a package for an ear-wearable device or accessory;
a removable element on the package; and
patient health information indicia affixed to the removable element.

2. The system of claim 1, wherein the removable element is configured to be detached from the package without using a tool.

3. The system of claim 1, wherein the package includes a void adjacent to an edge of the removable element to facilitate initiation of its removal from the package.

4. The system of claim 1, wherein the removable element includes a pull tab to facilitate its removal from the package.

5. The system of claim 1, further comprising a perforation border comprising a series of perforations, wherein the perforation border facilitates removal of the removable element from the package.

6. The system of claim 1, wherein corners of the removable element are rounded to facilitate its removal from the package.

7. The system of claim 1, further comprising a carrying case for the ear-wearable device or accessory designed to fit within the package, wherein the removable element is configured to be secured to the carrying case after its removal from the package.

8. The system of claim 1, further comprising the ear-wearable device or accessory.

9. The system of claim 1, wherein the removable element has an adhesive backing for reattachment to another surface after its removal from the package.

10. The system of claim 1, wherein the removable element has a hook or loop fastener backing for reattachment to another surface after its removal from the package.

11. The system of claim 1, wherein the removable element has a width of about 2.5 centimeters to about 8 centimeters, inclusive, and a length of about 5 centimeters to about 13 centimeters, inclusive.

12. The system of claim 1, wherein the patient health information indicia serve as an ear-wearable device or accessory identifier when affixed to the removable element.

13. The system of claim 1, wherein the removable element comprises a base portion and a separable portion, wherein the base portion comprises a first subset of non-patient health information indicia and the separable portion comprises a second subset of patient health information indicia, wherein the separable portion comprises one of a separable portion perforation border, a peel-off portion, or a scratch-off portion to facilitate removing the second subset of patient health information indicia from the removable element.

14. The system of claim 1, wherein the removable element comprises a patient health information card.

15. The system of claim 14, wherein the patient health information card includes one or more of: a patient's name, last four digits of his/her social security number, customer service contact information for the ear-wearable device, and a serial number of the ear-wearable device or accessory.

16. The system of claim 14, wherein the package includes a sleeve with a transparent outward-facing surface, configured to hold the patient health information card.

17. An ear-wearable device or accessory packaging system, comprising:
a package for an ear-wearable device or accessory;
a removable element integrated into the package;
patient health information indicia including a patient's name or a portion of his/her social security number affixed to the removable element; and
a perforation border comprising a series of perforations, wherein the perforation border facilitates removal of the removable element from the package without using a tool.

18. The system of claim 17, wherein the package includes a void adjacent to an edge of the removable element to facilitate initiation of its removal from the package.

19. The system of claim 17, further comprising a carrying case for the ear-wearable device or accessory, wherein the removable element is configured to attach to the carrying case after its removal from the package.

20. A method for using a packaging system for an ear-wearable device, comprising:
using patient health information indicia affixed to a removable element integrated into a package as a product identifier;
removing the removable element from the package by tearing along a series of perforations surrounding the removable element; and
either shredding the removable element or affixing it to a carrying case provided with the ear-wearable device.
